(19) United States
(12) Patent Application Publication
(10) Pub. No.: US 2016/0237110 A1
(43) Pub. Date: Aug. 18, 2016

(54) ACOUSTIC MICROREACTOR AND METHODS OF USE THEREOF

(71) Applicant: FloDesign Sonics, Inc., Wilbraham, MA (US)

(72) Inventors: Rudolf Gilmanshin, Framingham, MA (US); Bart Lipkens, Hampden, MA (US)

(21) Appl. No.: 15/044,309

(22) Filed: Feb. 16, 2016

Related U.S. Application Data
(60) Provisional application No. 62/116,897, filed on Feb. 16, 2015.

(51) Int. Cl. C07K 1/04; C07K 16/00; C12P 19/34

(52) U.S. Cl. CPC C07K 1/045; C12P 19/34; C07K 16/00

(57) ABSTRACT

Described herein are microreactors and methods of modifying support particles using acoustic standing waves. A liquid medium containing suspended support particles is flowed along a flow path through a channel, and an acoustic standing wave is applied to the channel to hold the suspended particles at a point in the channel. The held particles are then subjected to one or more reactions by flowing biochemical reactants through the channel. Unbound biological reactant is optionally washed from the channel. The reacted support particles can be released from the acoustic standing wave for further processing using, for example, flow cytometry or fluorescence microscopy.

### CROSS-REFERENCE TO RELATED APPLICATIONS

This application claims priority to U.S. Provisional Application 62/116,897 filed on Feb. 16, 2015, which is incorporated herein by reference in its entirety.

### FIELD OF THE DISCLOSURE

Described herein are devices and methods for manipulating components (e.g., particles) contained in a liquid medium using acoustic standing waves.

### BACKGROUND

Microfluidics systems are systems in which small volumes of fluid such as those containing particles are processed, e.g., mixed, separated, or moved, typically by flowing them through microchannels in a microfluidic device. Key applications for microfluidics include inkjet print heads, DNA chips, lab-on-a-chip technology, micro-propulsion, and micro-thermal technologies. Recently, flow cytometry has also been accomplished in a microfluidic environment.

Antibodies are used in immunoassays to detect and quantify antigens. While primary antibodies can be labeled by covalently attaching a label to the primary antibody and directly detecting the labeled primary antibody, it is usually advantageous to use indirect staining in which the primary antibody does not bear a directly detectable tag. These techniques include detection of unlabeled primary antibody with colloidal metal labeled antibodies or bacterial products which bind immunoglobulins of many mammalian species. Protein tags (Streptococcal Protein A, Streptococcal Protein G), primary antibodies, and other analyte specific reagents, such as nucleic acid probes derivatized with small molecules, including fluorescein, dinitrophenol, digoxigenin, and biotin can be detected using antibodies against the relevant tag. Biotinylated primary antibodies can also be detected using avidin tagged with a heavy metal. Secondary antibodies and avidin can react with more sites on the primary antibody than Protein A or G. This may lead to augmented signal when the former methods are employed, but complicates quantitation of antibody binding. The utility of Protein A is also limited to some extent by its ability to bind certain subclasses of immunoglobulin. This drawback can be circumvented by imposing a “bridging” antibody that binds to the primary antibody and is subsequently bound by labeled Protein A or Protein G. Such assays are often called “sandwich assays” because the analyte to be measured is bound between two primary antibodies. Other sandwich techniques include the avidin-biotin-peroxidase complex and peroxidase-anti-peroxidase methods, can be used to enhance the sensitivity of ultrastructural immunolabeling.

Primary antibodies can be very useful for the detection of biomarkers for diseases such as cancer, diabetes, Parkinson's disease and Alzheimer's disease and are also used for the study of multidrug-resistant therapeutic agents. Secondary antibodies are especially efficient in immunolabeling applications. In immunolabeling, the Fab domain of the primary antibody binds to an antigen and exposes its Fc domain to secondary antibody. Then, the Fab domain of the secondary antibody binds to the Fc domain of the primary antibody. Since the Fc domain of an antibody is constant within the same animal class, only one type of secondary antibody is required to bind many types of primary antibodies. This reduces the cost of labeling by only one type of secondary antibody, rather than labeling various types of primary antibodies.

What is needed is a method of reacting biological materials, particularly on a microfluidic scale, such as a process and device that improves the system of bringing together primary and secondary antibodies, where the secondary antibody can be a fluorescently labeled tag for the primary antibody.

### BRIEF SUMMARY

In one aspect, a method of biochemically modifying support particles comprises flowing a liquid medium containing suspended support particles along a flow path through a channel, applying an acoustic standing wave perpendicular to or at an angle to the flow path and holding the suspended support particles at a point in the channel to provide held support particles, flowing a first reactant solution containing a first biochemical reactant through the channel and allowing the first biochemical reactant to bind with the held support particles to provide modified held support particles, optionally flowing a first wash solution through the channel to remove unbound first biochemical reactant, optionally flowing a second reactant solution containing a second biochemical reactant through the channel and allowing the second biochemical reactant to bind with the held support particles to provide further modified held support particles, optionally flowing a second wash solution through the channel to remove unbound second biochemical reactant, and releasing the modified or further modified held support particles from the acoustic standing wave for further processing.

### BRIEF DESCRIPTION OF THE DRAWINGS

FIG. 1 is a schematic of a suspension array.

FIG. 2 is a schematic of a method of the present disclosure. In step 1, array particles are flowed into the flow chamber and held in the acoustic field. In step 2, a biological sample is flowed through the flow chamber for binding to the held array particles. In step 3, unreacted sample is removed by washing. In step 4, a secondary antibody solution is flowed through the reaction chamber for binding to the bound antigens. Step 5 is a second washing step to remove unbound secondary antibody. In step 6, the array particles are released for detection by flow cytometry. Other chemistries such as aptamers, oligonucleotides, streptavidin/biotin pairing and such can be equally implemented under this scheme.

FIG. 3 is a schematic of a flow channel that includes a layer of an acoustically transparent material in the walls of the channel.

The above-described and other features will be appreciated and understood by those skilled in the art from the following detailed description, drawings, and appended claims.

### DETAILED DESCRIPTION

Described herein are fluidic (e.g., microfluidic and macrofluidic) devices and methods suitable for performing biochemical reactions on particles suspended in a liquid medium. Specifically, an acoustic standing wave is produced perpendicular to or at an angle to a flow path through a channel (e.g., a microchannel or macrochannel) and the suspended particles are held at a point, such as the center, in the channel (e.g., in a node or anti-node of the standing wave) when the liquid medium is flowed through the channel. Once the particles are held within the channel by the acoustic standing wave, one or more solutions containing the biochemical reactant(s) are flowed through the channel, allowing for binding of the biochemical reactant(s) with the held particles. Advantageously, reactions can be performed on the held particles in the absence of wall effects from the channel. Optionally, unreacted biochemical reactant is removed from the channel by flowing a wash solution through the channel prior to subsequent biochemical reactions and/or release of the particles for detection. Once the biochemical reactions are completed, the particles are released from the standing wave and flowed out of the channel for further analysis such as by flow cytometry or optical microscopy. The system can be used for preparation of labeled particles (such as suspended arrays, vesicles, paramagnetic beads, etc.) in various formats. Fluorescently labeled samples are most useful, which can be used for analysis with various systems (e.g., plate readers, in addition to the flow cytometers and microscopes). This process allows for very low cost production of the fluorescently labeled materials as well as other reactants through the use of the acoustic standing wave in the microchannel or macrochannel configuration. In an advantageous aspect, the device is a microfluidic device and the channel is a microchannel.

As used herein, a microfluidic device is a device suitable for processing small volumes of fluid containing analytes, such as nanoliter and picoliter volumes of fluid. In general, microfluidic devices have dimensions of millimeters to nanometers, and comprise one or more micro channels, as well as inlet and outlet ports that allow fluids to pass into and out of the microfluidic device. A microfluidic chip, for example, is a microfluidic device into which a network of microchannels has been molded or patterned. In the devices described herein, a width of the microchannel typically corresponds to less than or equal to one half of the wavelength of the acoustic standing wave (λ/2) which is about 50 microns to about 2 millimeters, or less.

A macrofluidic device may also be utilized. A macrofluidic device will have a flow channel from greater than about 2 mm to about 10 mm. A macrochannel has a width greater than lambda, the wavelength of the acoustic standing wave, divided by two (λ/2).

In one aspect, the particles used in the presently described methods and devices are support particles, wherein the surface of the support particle is utilized in successive steps as a reactor for antigens and antibodies as well as fluorophores. As a result, when an acoustic standing wave is applied to the support particles in suspension, the axial acoustic radiation force (ARF) drives the support particles towards the acoustic standing wave pressure nodes. The axial component of the acoustic radiation force drives the support particles, with a positive contrast factor, to the pressure nodal planes, whereas support particles or other particles with a negative contrast factor are driven to the pressure anti-nodal planes. The radial or lateral component of the acoustic radiation force is the force that traps the support particles. The radial or lateral component of the ARF can be made larger than the combined effect of fluid drag force and gravitational force. For particles smaller than the wavelength of the acoustic standing wave, the drag force F_{D} can be expressed as:

$${\vec{F}}_{D} = 4 π {μ}_{f} {R}_{p} ( {\vec{U}}_{f} - {\vec{U}}_{p} ) [ \frac{1 + \frac{3}{2} \hat{μ}}{1 + \hat{μ}} ] ,$$

where U_{f} and Uₚ are the fluid and support material or particle velocity, Rₚ is the particle radius, μ_{f} and μₚ are the dynamic viscosity of the fluid and the support material or particle, and \hat{μ}=μₚ/μ_{f} is the ratio of dynamic viscosities. The buoyancy force F_{B} is expressed as:

$${F}_{B} = \frac{4}{3} π {R}_{p}^{3} ( {ρ}_{f} - {ρ}_{p} ) g .$$

For a support particle or particle to be trapped in the multi-dimensional ultrasonic standing wave, for example, the force balance on the cell must be zero, and, therefore, an expression for lateral acoustic radiation force F_{LRF} can be found, which is given by:

F_{LRF}=F_{D}+F_{B}.

For a cell of known size and material property, and for a given flow rate, this equation can be used to estimate the magnitude of the lateral acoustic radiation force.

The theoretical model that is used to calculate the acoustic radiation force is based on the formulation developed by Gor'kov. The primary acoustic radiation force F_{A} is defined as a function of a field potential U, F_{A}=−∇(U),

where the field potential U is defined as

$$U = {V}_{0} [ \frac{〈 {p}^{2} 〉}{2 {ρ}_{f} {c}_{f}^{2}} {f}_{1} - \frac{3 {ρ}_{f} 〈 {u}^{2} 〉}{4} {f}_{2} ] ,$$

and f₁ and f₂ are the monopole and dipole contributions defined by

$${f}_{1} = 1 - \frac{1}{{Λσ}^{2}} , {f}_{2} = \frac{2 ( Λ - 1 )}{2 Λ + 1} ,$$

where p is the acoustic pressure, u is the fluid particle velocity, Λ is the ratio of cell density ρₚ to fluid density ρ_{f}, σ is the ratio of cell sound speed cₚ to fluid sound speed c_{f}, Vₒ is the volume of the cell, and < > indicates time averaging over the period of the wave.

Gor'kov's theory is limited to particle sizes that are small with respect to the wavelength of the sound fields in the fluid and the particle, and it also does not take into account the effect of viscosity of the fluid and the particle on the radiation force. Additional numerical models have been developed for the calculation of the acoustic radiation force for a particle without any restriction as to particle size relative to wavelength. These models also include the effect of fluid and particle viscosity, and therefore are a more accurate calculation of the acoustic radiation force. The models that were implemented are based on the theoretical work of Yurii Ilinskii and Evgenia Zabolotskaya as described in AIP Conference Proceedings, Vol. 1474-1, pp. 255-258 (2012).

The ultrasonic transducer(s) may generate a multi-dimensional standing wave in the fluid that exerts a lateral force on the suspended particles (e.g., support material) to accompany the axial force. Multi-dimensional standing waves are described in detail in WO2014/124306, incorporated herein by reference for its disclosure of multi-dimensional standing waves. Typical results published in the literature state that the lateral force is two orders of magnitude smaller than the axial force. In contrast, the technology disclosed in this application provides for a lateral force to be of the same order of magnitude as the axial force. However, in certain embodiments described further herein, the device uses both transducers that produce multi-dimensional acoustic standing waves and transducers that produce planar acoustic standing waves. For purposes of this disclosure, a standing wave where the axial force is not the same order of magnitude as the lateral force is considered a “planar acoustic standing wave.” The lateral force of the total acoustic radiation force (ARF) generated by the ultrasonic transducer(s) of the acoustic standing wave is significant and is sufficient to overcome the fluid drag force at linear velocities of up to 1 cm/s, and to create tightly packed clusters.

In the present devices and methods, the suspended particles (e.g., support particles) are held in the flow path of a microchannel or macrochannel in order to perform reactions with biochemical reactants. By holding the particles in the channel using the acoustic standing wave and then flowing a first reactant solution containing a first biochemical reactant through the microchannel, the first biochemical reactant is allowed to bind with the held particles to provide modified held particles.

As used herein, the term bind generally means a specific noncovalent interaction, such as an antibody-antigen interaction. However, under certain conditions, binding may result in a covalent interaction such as that resulting from a chemical reaction between the held particle and the biochemical reactant. Furthermore, in other embodiments, binding can be a noncovalent non-specific interaction, e.g. staining with a fluorescent dye. Thus, when the term “bind”, “binds”, or “binding” is used herein, both noncovalent and covalent interactions are included in these terms. The term “bind”, “binds”, or “binding” also includes the case of extreme non-covalent binding (e.g. in biotin-streptavidin complex, which binding energy approaches a typical covalent reaction). Binding also includes multiple hydrogen bonds and ion bonds that are not explicitly covalent in water by energy, but approach it in water-free media.

Exemplary particles for biochemical modification include support particles such as microparticles, polymer beads, magnetic beads, superparamagnetic beads, and nanostrips. The support particles can include a receptor molecule such as a DNA oligonucleotide probe, an antibody, protein or peptide. The receptor molecule, for example, binds an antigen of interest. In certain aspects, the particles are microparticles or nanostrips that include an attached monoclonal antibody that specifically binds a cell surface marker such as a T-cell surface marker or a stem cell surface marker. For use in the present methods, the particles have diameters of about 1 to about 350 micrometers, such as about 300 micrometers. Without being held to theory, it is believed that the frequency of the acoustic standing wave determines the diameter of the particles that can be held in the wave. For example, for a 2 MHz wave, the particle size is about 1 to about 100 microns, for a 0.1 mHz wave, the particle size is about 20 to about 2000 microns, and for a 20 mHz wave, the particle size is about 0.2 to about 20 microns.

Exemplary microparticles for biochemical reactions include suspension array microspheres, and other shaped beads. Suspension array beads may be a plurality of polymeric beads wherein each type of microsphere bead has a unique identification based on variations in optical properties, typically fluorescence. The differently labeled microsphere beads further include a receptor molecule such as a DNA oligonucleotide probe, an antibody, protein or peptide. The receptor molecule, for example, binds an antigen of interest. Suspension array panels can be used to detect biomarkers for a range of maladies and bodily processes such as cancer and organ function. One suspension array panel can be used to detect biomarkers of inflammation such as TNF superfamily proteins, IFN family proteins, Treg cytokines, and MMPs (Bio-Plex Pro™ Human Inflammation Assays, Bio-Rad), biomarkers involved in diabetes, obesity, metabolic syndrome, cardiovascular disease (CVD), and hormonal control of metabolism and reproductive organs (Bio-Plex Pro™ RBM Human Metabolic and Hormone Assays, Bio-Rad), human matrix metalloproteinases (MMPs) and tissue inhibitors of matrix metalloproteinases (TIMPs) (Bio-Plex Pro™ Assays for human matrix metalloproteinases, Bio-Rad), chemokines from human biological samples (Bio-Plex Pro™ Human Chemokine Panel Assays, Bio-Rad), apoptosis biomarkers (Bio-Plex Pro™ RBM apoptosis multiplex assays, Bio-Rad), isotyping of immunoglobulins (Bio-Plex Pro™ RBM apoptosis multiplex assays, Bio-Rad), bacterial pathogens, for example. The Luminex® xMAP® system can be used for many applications including protein expression profiling, focused gene expression profiling, autoimmune disease, genetic disease, molecular infectious disease, and HLA testing. Probe-target hybridization is detected by detecting optically labeled targets which can determine the relative abundance of each target in the sample using flow cytometry, for example. Microsphere arrays have been successfully used for immunoassays, single nucleotide polymorphism (SNP), genotyping, bacterial signature detection, and detection of DNA or RNA viruses. Exemplary targets for hybridization include, cells, proteins, peptides, and nucleic acids, for example.

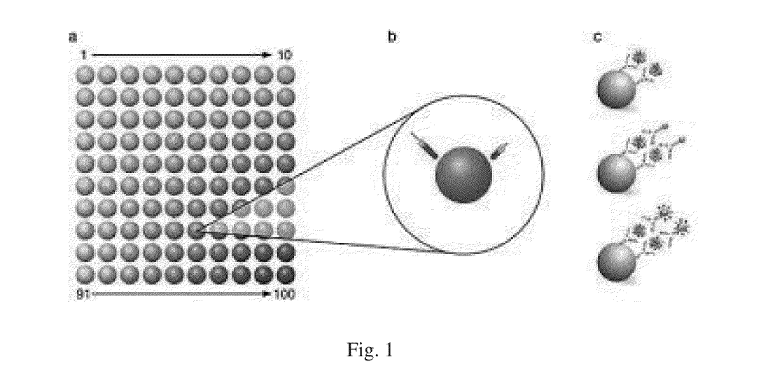

FIG. 1 is a schematic of a suspension array (a) in which multiple sensors can be suspended in solution. In this embodiment, every sensor bead carries a specific primary antibody or oligonucleotide and is coded by a combination of two fluorophores (b). In the presence of a target antigen or a nucleic acid motif, a secondary, fluorescent antibody or oligonucleotide can bind to the bead and generate a third color fluorescence. The fluorescence is then detected using flow cytometry or other detection methods.

Analysis of the microparticles in a suspension array is typically done by flow cytometry. Typically, the microparticles in the suspension array are diluted at least 10 fold, more typically at least 50 fold, most typically at least 100 fold before flow cytometry. An aqueous fluid that will not interfere with the optical analysis of the microparticles can be used to dilute the microparticles before analysis. Exemplary fluids for flow cytometry analysis include, for example, saline, phosphate buffered saline, Tris buffer, or culture media for mammalian cells. One of skill in the art will be able to select the degree of dilution and suitable fluids without undue experimentation.

Nanostrips are nanoscale test strips that enable clinical assays on blood samples, for example. In one aspect, a nanostrip can be held in an acoustic standing wave and the first biochemical reactant is a sample such as a blood sample that potentially contains an analyte that binds to the nanostrip. After exposure to the sample, the excess sample can be washed from the channel and the nanostrips collected for further analysis.

In one embodiment, a method of biochemically modifying particles (e.g., support particles) comprises flowing a liquid medium containing suspended particles along a flow path through a channel, applying an acoustic standing wave perpendicular or at an angle to the flow path and holding the suspended particles at a point in the channel to provide held particles, flowing a first reactant solution containing a first biochemical reactant through the channel and allowing the first biochemical reactant to react with the held particles to provide modified held particles, optionally flowing a first wash solution through the channel to remove unreacted first biochemical reactant, optionally flowing a second reactant solution containing a second biochemical reactant through the channel and allowing the second biochemical reactant to react with the held particles to provide further modified held particles, optionally flowing a second wash solution through the channel to remove unreacted second biochemical reactant, and releasing the modified or further modified held particles from the acoustic standing wave for further processing.

Exemplary biochemical reactants for biochemical modification of the particles include antibodies, aptamers, receptors, as well as streptavidin-biotin pairs, and polynucleotides including oligonucleotide probes and modified oligonucleotides (e.g., LNA or PNA). In specific embodiments, one or more of the biochemical reactants includes a detectable label such as a fluorescent label or other tag suitable for detection of the biochemical reactant.

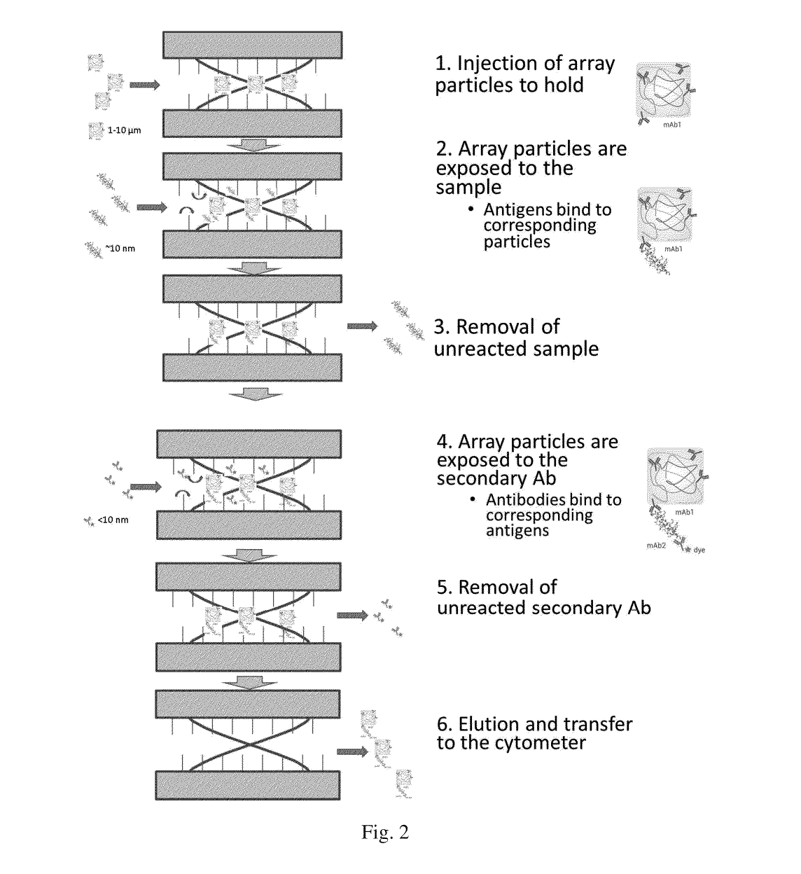

An embodiment of the method applied to a suspension array is shown in FIG. 2. In the first step, suspension array particles with an antibody reagent are flowed through the channel and held in an acoustic standing wave. Once the suspension array particles are held, in step two, a sample containing antigens is flowed through the cell allowing for binding of the antigens to the antibody reagent on the suspension array particles. Unreacted sample then can be removed in step three. In a fourth step, a solution containing fluorescent antibodies is flowed through the chamber which allows tagging of matching antigens. In step five the unreacted secondary antibody is washed away. Finally, in step six, the particles are released from the standing wave and transferred to a flow cytometer for detection. Thus, in one aspect, the first biochemical reactant is a sample suspected of containing an antigen and the second biochemical reactant is a labeled antibody that binds to the antigen.

Exemplary samples for the first biochemical reactant include blood, serum, plasma, cell culture (mammalian, yeast, bacterial), water samples, suspended tissues such as tumor samples, and the like.

In one aspect, an acoustophoretic device comprises one or more inlets in fluid communication with a first end of a channel and one or more outlets in communication with a second end of the channel. Fluid (containing particles and/or reactant solution) enters the channel through one or more inlets and exits the channel through the one or more outlets. In one aspect, the device includes one or more ultrasonic transducers arranged along the channel, wherein each ultrasonic transducer is paired with a reflector located on an opposite wall of the flow channel. The acoustic transducer and opposing reflector set up a resonant standing wave in the fluid in the channel. In another aspect, an angled field may be employed. An angled field is produced, for example, by placing the ultrasonic transducer-reflector pair at an angle to the flow field while maintaining the face of the ultrasonic transducer parallel to the face of the reflector. If the particles are flowed at an angle, the particles are all forced through the nodal plane, enhancing the chances to be trapped. The ultrasonic transducers can be driven by an oscillating, periodic, or pulsed voltage signal of ultrasonic frequencies. The frequency can be optimized for a specific range of particle sizes in the fluid.

Exemplary materials for the channel include quartz, glass, polydimethyl siloxane (silicone) and machined metal.

While there is no particular limit on the diameter of the channel, in certain aspects, the minimal diameter of a channel is half of an acoustic wavelength, or 0.4 mm for a 2 MHz wave. The channel may be wider when it includes multiple wavelengths. The volume of the flow path is, for example, 0.05 to 100 mL, specifically 0.05 to 0.5 mL.

Three-dimensional (3-D) acoustic standing waves can be produced from one or more piezoelectric transducers, where the transducers are electrically or mechanically excited such that they move in a “drumhead” or multi-excitation mode rather than a “piston” or single excitation mode fashion. Operation in the piston” or single excitation mode produces planar waves. Through this manner of wave generation, a higher lateral trapping force is generated than if piezoelectric transducer is excited in a “piston” mode where only one large standing wave is generated. Thus, with the same input power to a piezoelectric transducer, the three-dimensional acoustic standing waves can have a higher lateral trapping force compared to a single acoustic standing wave.

In operation, the acoustic radiation force (F_{ac}) acts on the suspended particles, pushing them to the center of the chamber wherein they are held by the acoustic standing wave.

The acoustophoretic device includes one or more transducers. In one aspect, the transducer is made of a piezo-electric material such as lead zirconate titanate (PZT).

Cylindrical transducers may also be utilized in the microchannel or macrochannel. Practical lengths of the transducers are 10-100 mm. Practical widths of the transducers are less than 20 mm for a flat system.

The transducer, e.g., a piezoelectric transducer, can be driven by a pulsed voltage signal. This pulsed pattern can be repeated according to a repetition rate or period. In another implementation, a piezoelectric transducer can be driven by a pulsed voltage signal that is a square or saw-toothed wave.

The ultrasonic frequencies can be in the range from 1 kHz to 100 MHz, with amplitudes of 1-100 of volts, normally acting in the tens of volts. The ultrasonic frequencies can be between 200 kHz and 3 MHz. The ultrasonic frequencies can be between 1 and 3 MHz. The ultrasonic frequencies can be 200, 400, 800, 1000 or 1200 kHz. The ultrasonic frequencies can be between 1 and 5 MHz. A reflector can be located opposite to the transducer, such that an acoustic standing wave is generated in the liquid medium. The acoustic standing wave can be oriented perpendicularly to the direction of the mean flow in the flow channel. The acoustic field exerts an acoustic radiation force, which can be referred to as an acoustophoretic force, on the suspended phase component.

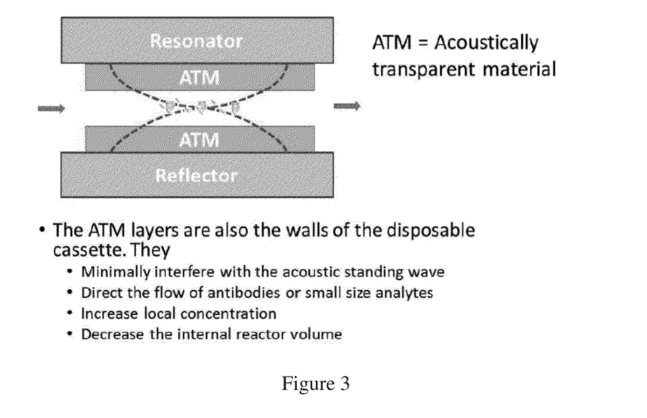

In one aspect, the channel, e.g., a microchannel, comprises an acoustically transparent material that is in communication with the flow path. The use of an acoustically transparent material for the channel provides the advantages of minimal interference with the acoustic standing wave, directing the flow of small size analytes through the channel, increasing the local concentration of the held particles as well as the biochemical reactants, and effectively decreasing the internal reactor volume. Without being held to theory, it is believed that by forming the channel from an acoustically transparent material, will allow for “squeezing” of the cells in the center of the flow path, thus providing a smaller effective volume for reaction without disturbing the acoustic pattern. (FIG. 3)

In one aspect, the acoustically transparent material is oriented polypropylene or low density polyethylene. The acoustic contrast factor of the material should be similar to water to avoid reflection losses at the interface with water-like fluids.

Further processing of the particles may include flowing the particles to a detector module. In one embodiment, the detector module is a flow cytometer having one or more lasers, optics, photodiodes, a photomultiplier tube, and digital signal processing to perform simultaneous, discrete measurements of fluorescent microspheres. In one embodiment, three avalanche photodiodes and a high sensitivity photomultiplier tube (PMT) receive photon signals from the microspheres. The detector module in this example digitizes the waveforms and delivers the signals to a digital signal processor (DSP). The detector module works with a host computer to perform multiplexed analysis simultaneously by using the flow cytometer and digital signal processor to perform real-time analysis of multiple microsphere-based assays. Because a flow cytometer has the ability to discriminate different particles on the basis of size and/or fluorescence emission color, multiplexed analysis with different microsphere populations is possible. Differential dyeing microspheres, emitting light at two different wavelengths, allows aggregates to be distinguished and permits discrimination of, in one embodiment, up to about 25 different sets of microspheres, in another embodiment up to about 100 different sets of microspheres, and in yet another embodiment more than 100 different sets of microspheres. Several control beads are used in every analysis to ensure quality control of the results.

In another embodiment, the detector module is an optical microscope capable of fluorescence detection.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of biochemically modifying support particles, comprising
flowing a liquid medium containing suspended support particles along a flow path through a channel,
applying an acoustic standing wave perpendicular or at an angle to the flow path and holding the suspended support particles at a point in the channel to provide held support particles,
flowing a first reactant solution containing a first biochemical reactant through the channel and allowing the first biochemical reactant to bind with the held support particles to provide modified held support particles,
optionally flowing a first wash solution through the channel to remove unbound first biochemical reactant,
optionally flowing a second reactant solution containing a second biochemical reactant through the channel and allowing the second biochemical reactant to bind with the held support particles to provide further modified held support particles,
optionally flowing a second wash solution through the channel to remove unbound second biochemical reactant, and
releasing the modified or further modified held support particles from the acoustic standing wave for further processing.

2. The method of claim 1, wherein the channel is a microchannel and a width of the microchannel is about 50 micrometers to about 2 millimeters.

3. The method of claim 1, wherein the channel is a macrochannel and a width of the microchannel is greater than about 2 millimeters to about 50 millimeters.

4. The method of claim 1, wherein the acoustic standing wave is a multi-dimensional acoustic standing wave.

5. The method of claim 1, wherein the acoustic standing wave is generated by an ultrasonic transducer-reflector pair, wherein the ultrasonic transducer and the reflector are located on opposite walls of the flow channel.

6. The method of claim 1, wherein the channel comprises an acoustically transparent material that is in communication with the flow path.

7. The method of claim 6, wherein the acoustically transparent material is oriented polypropylene or low density polyethylene.

8. The method of claim 6, wherein the acoustically transparent material increases the local concentration of the held support particles.

9. The method of claim 1, wherein the volume of the flow path is 0.05 to 100 mL.

10. The method of claim 1, wherein the support particles are microparticles, nanostrips, magnetic beads, paramagnetic beads, or polymer beads.

11. The method of claim 10, wherein the support particles comprise a receptor molecule that binds an antigen.

12. The method of claim 10, wherein the particles are microparticles or nanostrips having attached thereto a monoclonal antibody that specifically binds a cell surface marker.

13. The method of claim 12, wherein the cell surface marker is a T-cell surface marker or a stem cell surface marker.

14. The method of claim 1, wherein the support particles form a suspension array.

15. The method of claim 1, wherein the first biochemical reactant and the second biochemical reactant are each independently antibodies, aptamers, receptors, streptavidin-biotin pairs, or polynucleotides.

16. The method of claim 15, wherein the first biochemical reactant is a sample suspected of containing an antigen, and the second biochemical reactant is a labeled antibody that binds to the antigen.

17. The method of claim 1, wherein the first biochemical reactant or the second biochemical reactant comprises a detectable label.

18. The method of claim 1, wherein releasing the modified or further modified held particles from the acoustic standing wave for further processing comprises flowing the particles to a detector module.

19. The method of claim 18, wherein the detector module is a flow cytometer or a fluorescence microscope.

20. The method of claim 1, wherein the channel forms part of a microfluidic chip.
